(19) United States
(12) Patent Application Publication
(10) Pub. No.: US 2020/0411172 A1
(43) Pub. Date: Dec. 31, 2020

(54) INTEGRATED VALUE BASED CARE RETURN ON INVESTMENT SYSTEM

(71) Applicant: Walter M Groteke, Safety Harbor, FL (US)

(72) Inventors: Eric K Groteke, Safety Harbor, FL (US); Walter M Groteke, Safety Harbor, FL (US)

(21) Appl. No.: 16/913,236

(22) Filed: Jun. 26, 2020

Related U.S. Application Data
(60) Provisional application No. 62/866,786, filed on Jun. 26, 2019.

(51) Int. Cl. G16H 40/20; G06Q 10/10; G06Q 10/06; G06F 16/25; G06F 21/62; G06N 5/04

(52) U.S. Cl. CPC G16H 40/20; G06Q 10/10; G06Q 10/06375; G06Q 20/127; G06F 21/6254; G06N 5/04; G06F 16/254

(57) ABSTRACT

An integrated value based care return on investment is disclosed.

### BACKGROUND OF THE INVENTION

Problem Solved: Currently clinical and sub-clinical process decisions have historically relied on the symptom(s) generators and diagnosing causality much in alignment with the practice of the medical diagnosis arts. In an ever-evolving environment, judgment and experimentally-developed predictive models are utilized by the healthcare end-users to utilize the information currently at hand to offer guidance to patients and make course of treatment decisions unilaterally with little evidence of causal relation to the direct and indirect risk factors that may predispose an individual or animal to a catastrophic health event or claim. The problem is compounded by these risk factors that are asymptomatic or “hidden” to the individual or animal and their identification vary from one healthcare provider to another. Employers/payers/Governments/health systems are bearing the brunt of ever increasing healthcare expenditures of their employees with little access to the direct and indirect risk factor data and how they can leverage it to improve individual's health and productivity while saving money and improving efficiencies. There is a lack of transparency between payers and employers, as employers are being prevented from having visibility to sensitive employee healthcare data and are therefore prohibited in being involved in the healthcare decisions of their workforce.

### BRIEF DESCRIPTION OF THE DRAWINGS

For exemplification purposes, and not for limitation purposes, aspects, embodiments or examples of the invention are illustrated in the figures of the accompanying drawings, in which:

FIGS. 1A-1B illustrate a diagram of how one version of the invention produces a report.

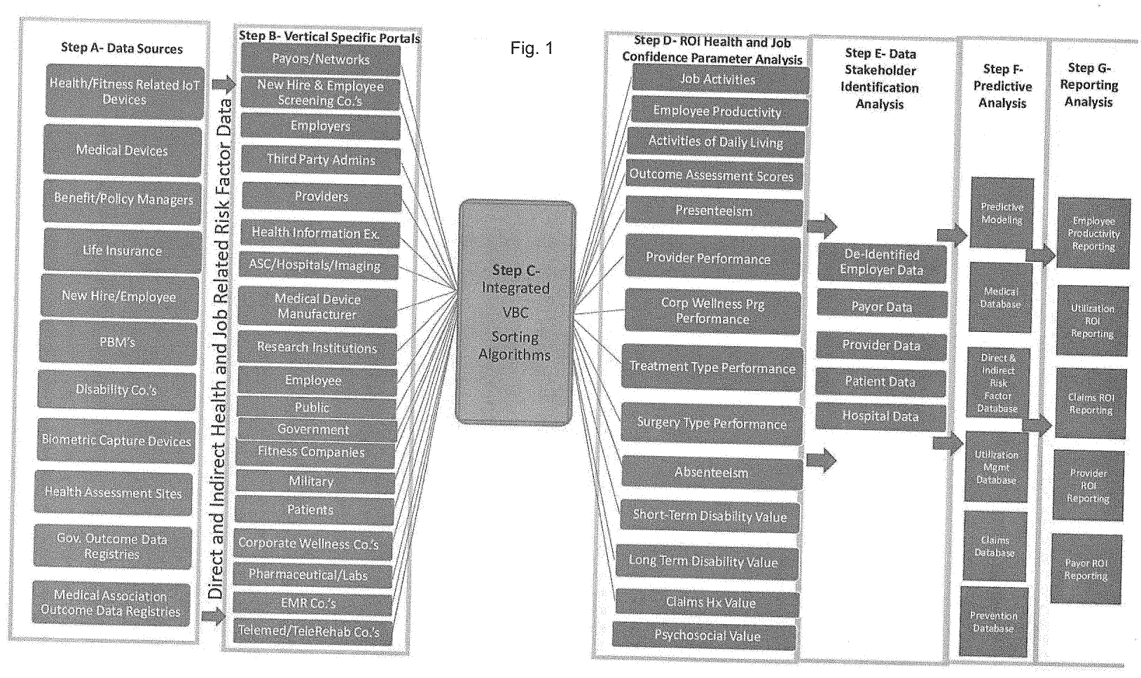

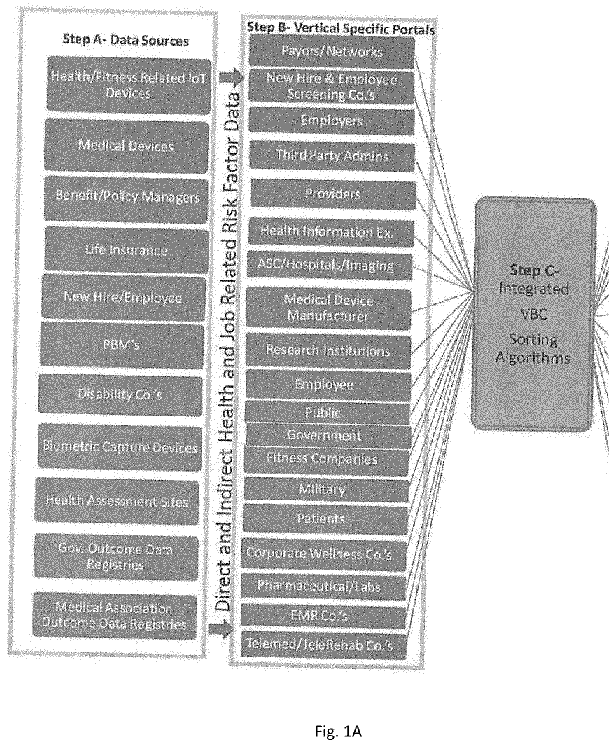

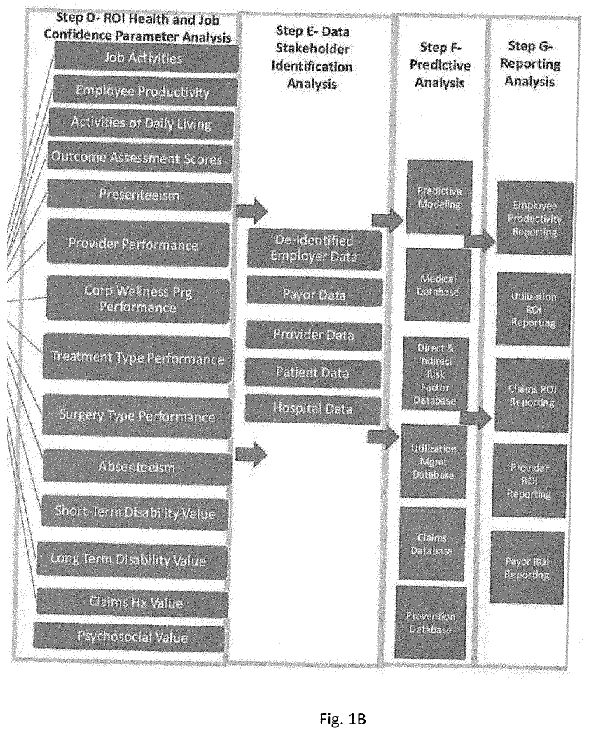

### DETAILED DESCRIPTION OF THE INVENTION

As stated above, currently clinical and sub-clinical process decisions have historically relied on the symptom{s) generators and diagnosing causality much in alignment with the practice of the medical diagnosis arts. In an ever-evolving environment, judgment and experimentally-developed predictive models are utilized by the healthcare end-users to utilize the information currently at hand to offer guidance to patients and make course of treatment decisions unilaterally with little evidence of causal relation to the direct and indirect risk factors that may predispose an individual or animal to a catastrophic health event or claim. The problem is compounded by these risk factors that are asymptomatic or “hidden” to the individual or animal and their identification vary from one healthcare provider to another. Employers/payers/Governments/health systems are bearing the brunt of ever increasing healthcare expenditures of their employees with little access to the direct and indirect risk factor data and how they can leverage it to improve individual's health and productivity while saving money and improving efficiencies. There is a lack of transparency between payers and employers, as employers are being prevented from having visibility to sensitive employee healthcare data and are therefore prohibited in being involved in the healthcare decisions of their workforce. The invention claimed here solves this problem.

Our method gathers vertical specific healthcare related data and organizes, quantifies, identifies and reports it in vertical specific meaningful return on investment reporting.

The claimed invention differs from what currently exists. Other value based care methods, processes or computer systems do not gather, organize, and quantify and vertically specific report direct and indirect risk factors as they relate to return on investment reporting.

This invention is an improvement on what currently exists. Other value based care methods, processes or computer systems do not gather, organize, and quantify and vertically specific report direct and indirect risk factors as they relate to return on investment reporting.

Also, it can produce The resultant data can create the framework/anatomy of return on investment strategies of direct and indirect risk factors impact on a specific individual's or population's for a desired outcome. E. g. aggregated return on investment data may result in specific healthcare delivery and/or payment strategies to take place based on summation of confidence impact parameter values.

The Version of the Invention Discussed Here Includes:

1. Step A—Data Sources

2. Step B—Vertical Specific Portals

3. Step C—Integrated Value Based Care Sorting Algorithms

4. Step D—Return-On-Investment Health and Job Confidence Parameter Analysis

5. Step E—Data Stakeholder Identification Analysis

6. Step F—Predictive Analysis

7. Step G—Reporting Analysis

Relationship Between the Components:

Direct and indirect risk factor data is collected from the various sources listed in diagram 1 then segregated into their appropriate classification via the vertical specific portal for analysis with value based care algorithms based on stakeholder end user.

1. Determining at least one source of stakeholder data and one intended use of stakeholder data, wherein each data element is associated with an impact parameter for the health or business-related conclusion:

2. For each data element, generating a confidence parameter as a function of the health or business-related conclusion; and,

3. Determining an overall level of confidence parameter as a function of each of the confidence parameters and the associated data impact values on that region(s) of the body and its intended stakeholder reporting needs.

How the Invention Works:

The end-user subscribes to access their stakeholder specific portal via a secure network connection. Data is either manually or automatically exchanged with the integrated value-based care tracking protocols, Acquired data is processed through an integrated customer re source management system to prepare/format/process data for the necessary stakeholder's reporting needs. Acquired data is then transformed into a compliant stakeholder database (e.g. De-identification of data, if required), then acquired data is processed to a health related or business conclusion report with a focus on direct and indirect health risk factors and their impact on the stakeholder's health related business decisions. The operation is conducted through a health information exchange and tele-health portal that contains health and business related direct and indirect risk factors related to data processing, enabling users to select specific body regions, answer a series of questions against evidence-based historical values which in turn produce a comparison against a large population of such data. Integrated value-based care reporting of their positive findings can be shared as a communication tool to effectuate value-based care and direct to employer/payer type of relationships/agreements. The use of reporting health and business data will facilitate a more effective method for a more accurate and real-time pay for performance healthcare model.

The present invention provides a method and system for collaboration and evaluation of health and business-related data to be shared, reported in a complaint value-based reporting system between all stakeholders involved.

The present invention may be applied either retrospectively to evaluate a prior health or business-related conclusions or prospectively to evaluate one or more hypothetical business related or health related conclusions. A method or system, which may be implemented as a back end at a node coupled to a customer resource management and health information network such as the health information exchanges and electronic medical health records, converts raw medical data into a report based on the stakeholders needs.

The method also performs a business and health related present and predictive conclusion analysis and stakeholder specific reporting process to evaluate one or more business or health related conclusions with respect to the selected body regions involved.

How to Make the Invention:

The present invention provides a method and system for integrating stakeholder data through a value based care interface method for the purpose of categorizing/quantifying the data into bodily regions and risk factor stratification for the purpose of sharing, collaborating and predicting future health related events and tracking key performance clinical, employer, payer and case/utilization management metrics. The present invention may be applied either retrospectively to evaluate a prior conclusion, evaluate a present clinical or sub-clinical scenario or prospectively to evaluate in a predictive value-based care delivery program.

According to one embodiment, a medical analysis site is hosted to a HIPAA compliant shared information portal. Clients including the aforementioned stakeholders interact with the source of the data collection system/program or device via a compliant Internet connection to the value-based care tracking portal for medical, quantitative, benchmarking, key performance index and predictive analysis. The integrated value-based care tracking portal/site receives input data from consented stakeholders either in the form of raw data or medical records, which are further processed based on their intended use by the various stakeholders. An example of this is when an employee seeks care that a self-insured employer is paying for the employer cannot legally view the medical records of their employee due to HIPAA and PHI statutes. In this scenario, the value-based care tracking system will de-identify that patient data and enable the employer to view the result of the treatment in report such as a return on investment productivity report showing the aggregate impact of their employee population healthcare and corporate wellness program. This shared type of information will enable the employer to make more realtime and cost-effective decisions based on the care that they are paying for on their employee population. The integrated value based care tracking analysis site/includes a front-end sub-portal (e.g., a World Wide Web server) for providing a graphical user interface (“GUI”) for clients to interact with the site, a core engine, which perform at least one process for analyzing and evaluating incoming stakeholder data in order to appropriately and compliantly map that data for benchmarking and predictive decision making and reporting to another stakeholder's intended use. With medical conclusions, predictive conclusions and a patient record database, which shares normalized medical data relating to medical histories and assessment findings of patients.

The integrated value based care tracking analysis site shares a predefined set of clinical and subclinical direct and indirect risk factors, as described above that represent normal events in an IoT device, medical device, fitness or wellness program, medical history including biometrics, direct and indirect risk factors to the human body, comorbidities, symptoms, treatments, clinical conclusions, laboratory tests, chronological factors, demographic factors, assessments, outcomes assessment scores, other health related scores intended to standardize care, diagnoses, utilization management, claims data and treatment plan data, etc.

According to one embodiment of the present invention, the core engine includes a processor and relational databases, which further includes a customer service relationship system, clinical and sub-clinical health and job related essential element database, a medical phrase database, a chronological rules database and a medical knowledge rules database, predictive analytics rules and database, telemedicine, tele-rehabilitation, electronic medical records system and various API's for interoperability to the necessary stakeholder and source of data acquisition users. The integrated value-based care tracking system database maps each incoming data point to a monetary value, employer related value, clinical or sub-clinical value, claims value to arrive at a compliant report based on the stakeholders desire. Analysis of at least one clinical or business-related conclusion uses a membership confidence function and a criterion impact parameter, based on available research, datamining and other relevant sources of data. The membership confidence function relates a degree of confidence that a particular essential element points to a particular clinical or business conclusion as a function of clinical or sub-clinical assessment that is consistent with evidence-based research or historical claims/utilization data.

How to Use the Invention:

The end-user subscribes to access their stakeholder specific portal via the internet. Data is either manually or automatically exchanged with the integrated value-based care tracking system. Acquired data are that are processed through an integrated customer resource management system to prepare/format/process data for the necessary stakeholders reporting needs. Acquired data is then transformed into a compliant stakeholder database (e.g. De-identification of data, if required), then acquired data is processed to a health related or business conclusion report with a focus on direct and indirect health risk factors and their impact on the stakeholders health related business decisions. The operation is conducted through a health information exchange and tele-health portal that contains health and business related direct and indirect risk factors related data processing, enabling users to select a specific body region, answer a series of questions against evidence-based historical values which in turn produce a comparison against a large population of such data. Integrated value-based care reporting of their positive findings can be shared as a communication tool to effectuate value-based care and direct to employer/payer type of relationships/agreements.

Additionally: Data gathering can be manual and resultant stakeholder reports can be derived via manual calculations/formulas to derive confidence impact values for vertical specific use of data.

Also, it can create: The resultant data can create the framework/anatomy of return on investment strategies of direct and indirect risk factors impact on a specific individual's or populations for a desired outcome. E.g. aggregated return on investment data may result in specific healthcare delivery and/or payment strategies to take place based on summation of confidence impact parameter values,

What is claimed is:

1. An integrated value based care return on investment system as described herein.
